(19) United States
(12) Patent Application Publication
(10) Pub. No.: US 2011/0137134 A1
(43) Pub. Date: Jun. 9, 2011

(54) METHOD AND SYSTEM FOR ADMINISTERING AN ANAESTHETIC

(76) Inventors: Thomas Hemmerling, Quebec (CA); Emile Salhab, Antelias (LB); Guillaume Trager, Antony (FR); Stephane Deschamps, Montreal (CA); Pierre A. Mathieu, Montreal (CA)

(21) Appl. No.: 12/448,924

(22) PCT Filed: Jan. 17, 2008
(86) PCT No.: PCT/CA2008/000103

§ 371 Date: Aug. 23, 2010

Related U.S. Application Data
(60) Provisional application No. 60/885,309, filed on Jan. 17, 2007.

(51) Int. Cl. A61B 5/00

(52) U.S. Cl. 600/301

(57) ABSTRACT

A method and system for objectively scoring intra-operative pain during general anaesthesia based on the patient's mean arterial pressure and heart rate. The index is used for closed-loop control of the intra-operative analgesia through adjustment of the drug infusion level according to fuzzy logic. It is further displayed along with other components of anaesthesia and important patient data on a monitoring display for presentation to medical staff.

### CROSS REFERENCE TO RELATED APPLICATIONS

This application claims priority on U.S. Provisional Application No. 60/885309, filed on Jan. 17, 2007 and which is herein incorporated by reference in its entirety.

### FIELD OF THE INVENTION

The present invention relates to a method and system for administering an anaesthetic, in particular for calculating an objective index representative of the intra-operative pain level using fuzzy-logic algorithms.

### BACKGROUND OF THE INVENTION

As well known in the art, anaesthesia is a reversible pharmacological state that aims at avoiding pain and protecting the patient undergoing surgery from physiological perturbations resulting from surgical manipulation. Anaesthesia can be general, in which case the patient loses consciousness as a result of administration of anaesthetic drugs, or local where only the area of the body, where surgery will be performed, is concerned. During general anaesthesia the patient goes through three consecutive phases: muscle relaxation, analgesia and hypnosis, which represent the three principal components of anaesthesia. Muscle relaxation is induced with muscle relaxants to ease the access to internal organs and to decrease involuntary muscle reflex responses to surgical stimulations. Hypnosis is associated with unconsciousness and absence of postoperative recall of events that occurred during surgery (intra-operative). Analgesia relates to pain relief and is reached through administration of drugs that decrease or suppress pain (analgesics) by intravenous injection or inhalation. Typical analgesics include sufentanil, alfentanil and remifentanil.

To achieve adequate anaesthesia and compensate the effect of surgical manipulation while maintaining the vital functions of the patient, anaesthesiologists must regularly adjust the settings of several drug infusion devices based on monitor readings of the patient's vital signs (e.g. breathing, blood pressure), which are compared to predetermined intra-operative target values. Although objective measures for muscle relaxation and hypnosis have been developed to determine the amount of anaesthetic medication that should be given to a patient, there is no specific measure of pain when the patient is unconscious since referring to “pain” during general anaesthesia is debatable. Indeed, the International Association for the Study of Pain defines pain as an “unpleasant sensory and emotional experience associated with actual or potential tissue damage”. However, clinical signs of pain such as tearing, pupil reactivity, eye movement and grimacing are partially suppressed by anaesthetic agents such as muscle relaxants. As a result, the anaesthesiologist must act subjectively during the surgical procedure, using his/her judgement, experience and surgical variables such as the degree of a surgical stimulus that is likely to cause pain to evaluate the level of pain suffered by the patient.

The prior art reveals that most accepted measures for assessing pain level during general anaesthesia are the Heart Rate (HR) and Mean Arterial Pressure (MAP). Indeed, changes in MAP or HR during surgery can be induced by pain as analgesics used for pain control are known to effectively block MAP or HR changes. Still, these two parameters can be influenced by other factors such as bleeding and subsequent decrease of blood pressure. Moreover, there is at present no method for objectively and quantitatively scoring intra-operative pain combining both MAP and HR measurements. Also, there is currently no means for integrating and reflecting the principal components of anaesthesia described above in a user friendly manner, thus facilitating decision making and decreasing the practitioner's workload.

### SUMMARY OF THE INVENTION

In order to address the above and other drawbacks, there is provided in accordance with the present invention a method for displaying an indicator of a current pain level of a patient being administered an analgesic. The method comprises providing a display device, measuring a current mean arterial pressure and heart rate of the patient, deriving the indicator from the measured current mean arterial pressure and heart rate, and displaying the derived indicator on the display device.

In accordance with the present invention, there is also provided a system for displaying an indicator representative of a current pain level of a patient being administered an analgesic. The system comprises a monitoring subsystem for measuring a current mean arterial pressure and heart rate of the patient and deriving the indicator from the measured mean arterial pressure and heart rate, and a display device coupled to the monitoring subsystem for displaying the derived indicator.

Still in accordance with the present invention, there is also provided a system for displaying a current state of anaesthesia of a patient undergoing surgery. The system comprises a first subsystem for measuring a current anaesthetic depth in the patient, a second subsystem for monitoring a current level of muscular relaxation in the patient, a third subsystem for deriving an indicator representative of a current pain level of the patient, and a display device coupled to the first, second, and third subsystems for simultaneously displaying the current anaesthetic depth, the current level of muscular relaxation and the derived indicator.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 is a schematic diagram of a system for monitoring a patient during surgery in accordance with an illustrative embodiment of the present invention;

FIG. 2 is a schematic diagram of a closed-loop anaesthesia control system in accordance with an illustrative embodiment of the present invention;

FIG. 3 is a table used for computation of an intra-operative pain index using fuzzy logic in accordance with an illustrative embodiment of the present invention;

FIG. 4 is a table used for adjusting the level of infusion of an anaesthetic agent during surgery through fuzzy logic in accordance with an illustrative embodiment of the present invention;

FIG. 5 is a flow chart of a closed-loop control algorithm used to adjust the level of infusion of an anaesthetic agent during surgery through fuzzy logic in accordance with an illustrative embodiment of the present invention;

FIG. 6 is a screen capture of a monitoring display during the patient setup phase in accordance with an illustrative embodiment of the present invention;

FIG. 7a is a screen capture of a monitoring display during the induction phase in accordance with an illustrative embodiment of the present invention;

FIG. 7b is a screen capture of a monitoring display during the target setup phase in accordance with an illustrative embodiment of the present invention; and

FIG. 8 is a screen capture of a monitoring display during the patient maintenance phase in accordance with an illustrative embodiment of the present invention.

### DESCRIPTION OF ILLUSTRATIVE EMBODIMENTS

The present invention is illustrated in further details by the following non-limiting examples.

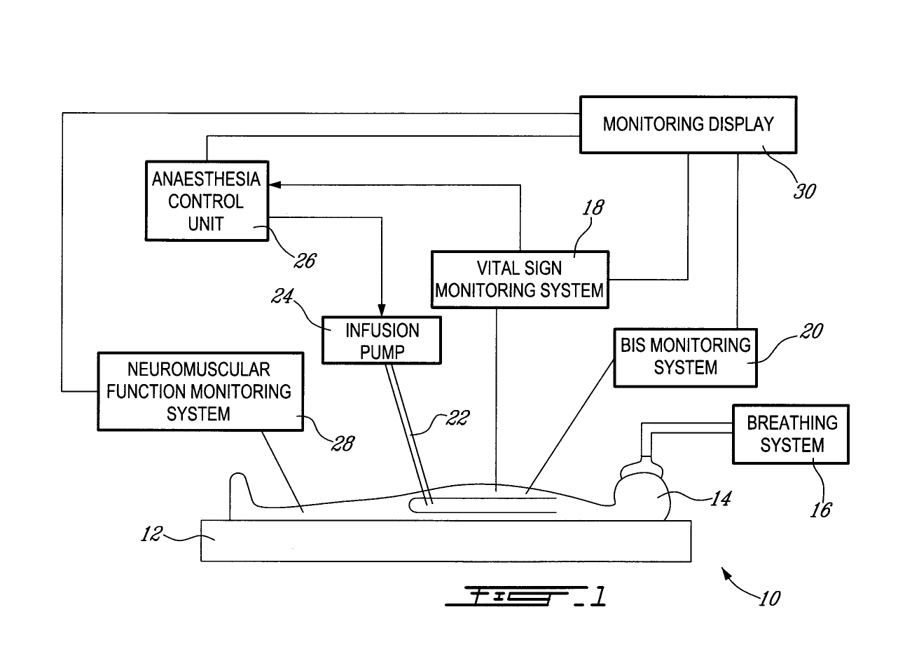

Referring to FIG. 1, and in accordance with an illustrative embodiment of the present invention, a system for patient monitoring and assistance during surgery, generally referred to using the reference numeral 10, will now be described. The system comprises an operating table 12, on which the patient 14 is lying during the surgery procedure. To maintain an open airway and regulate breathing within acceptable parameters, the unconscious patient 14 is connected to a breathing system 16 that replaces spontaneous breathing. In order to allow for a controlled induction of, maintenance of, and emergence from general anaesthesia, the patient 14 is also monitored using a vital sign monitoring system 18. Measured parameters include Heart Rate (HR) and heart rhythm, blood pressure (BP), pulse oxymetry (amount of oxygen in the blood), respiratory rate, and temperature. A Bispectral (BIS) monitoring system 20 is also used to measure the BIS index, which is representative of hypnosis i.e. the depth of anaesthesia.

Still referring to FIG. 1, liquid anaesthetic agents are administered intravenously from a delivery system, e.g. an infusion pump 24, to the patient 14 through a tube 22 such as a catheter. The infusion pump 24 is controlled by an anaesthesia control unit 26 to accurately monitor and regulate the dosage of analgesic administered to the patient 14 for pain management. The control unit 26 receives information from the vital sign monitoring system 18, and more specifically the patient's blood pressure and heart rate, and uses this information to derive an indicator or index representative of the patient's pain level, i.e. the Analgoscore. From this index, the control unit 26 determines how the level of analgesic administered to the patient 14 should be adjusted. As will be apparent to a person skilled in the art, the infusion pump 24 may be illustratively controlled by the anaesthesiologist rather than the control unit 26. In the latter case, once the Analgoscore is computed, the anaesthesiologist will vary accordingly the rate of infusion of the anaesthetic agent being administered to the patient by manually adjusting the infusion pump 24. A neuromuscular function monitoring system 28 also measures the level of neuromuscular blockade, which is representative of muscle relaxation. All three components of anaesthesia (pain, hypnosis, muscle relaxation) are displayed on a monitoring display 30 along with other important data related to the patient's physiological state during surgery.

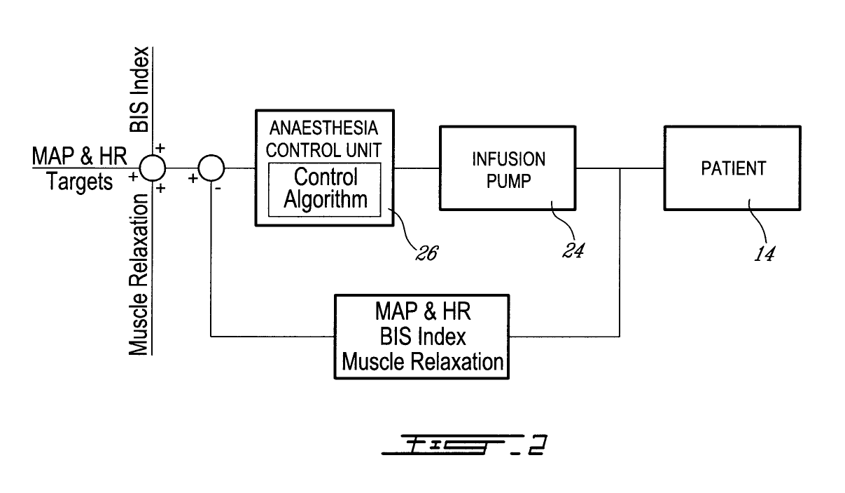

Referring now to FIG. 2 in addition to FIG. 1, infusion of an analgesic may be illustratively closed-loop controlled through a control algorithm invoked by the anaesthesia control unit 26, as discussed herein below. Before the first surgical incision, the anaesthesia level along with target values of BP and HR to be achieved in the patient 14 during surgery are initially established by the anaesthesiologist according to the patient's health record and in this case fed into the anaesthesia control unit 26 for implementation of the control algorithm. At the outset of anaesthesia, anaesthetic agents (e.g. muscle relaxants, analgesics, sedative agents) are thus infused through the infusion pump 24 to induce unconsciousness in the patient 14. Once this state has been reached, the surgical procedure can begin and the patient's vital signs (BP and HR) are monitored using the vital sign monitoring system 18. Two components of BP are typically measured: the systolic pressure (SP) and diastolic pressure (DP), which respectively represent the BP when the heart contracts and relaxes. The Mean Arterial Pressure (MAP), which represents the patient's average BP, is further computed from these two components as follows:

$$MAP = \frac{2 DP + SP}{3} \qquad ( 1 )$$

The anaesthesia control unit 26 then computes a first Analgoscore value using MAP and HR measurements determined periodically (e.g. once every minute) and invokes a control algorithm, which identifies whether changes in the dosage of the infused analgesic are required, according to the computed index of patient intra-operative pain. The information is then fed to the infusion pump 24, which will make necessary adjustments to the infusion. Alternatively, as mentioned herein above, the adjustments may be directly carried out by the anaesthesiologist, without implementation of the control algorithm. As can be seen from FIG. 2, the control unit 26 also illustratively receives inputs related to the other components of anaesthesia, namely the patient's BIS index and level of muscle relaxation, which are respectively measured by the BIS monitoring system 20 and the neuromuscular function monitoring system 28. These inputs will allow for control of the dosage of other anaesthetic agents, such as muscle relaxants and sedative agents, in addition to controlling the infusion of analgesic.

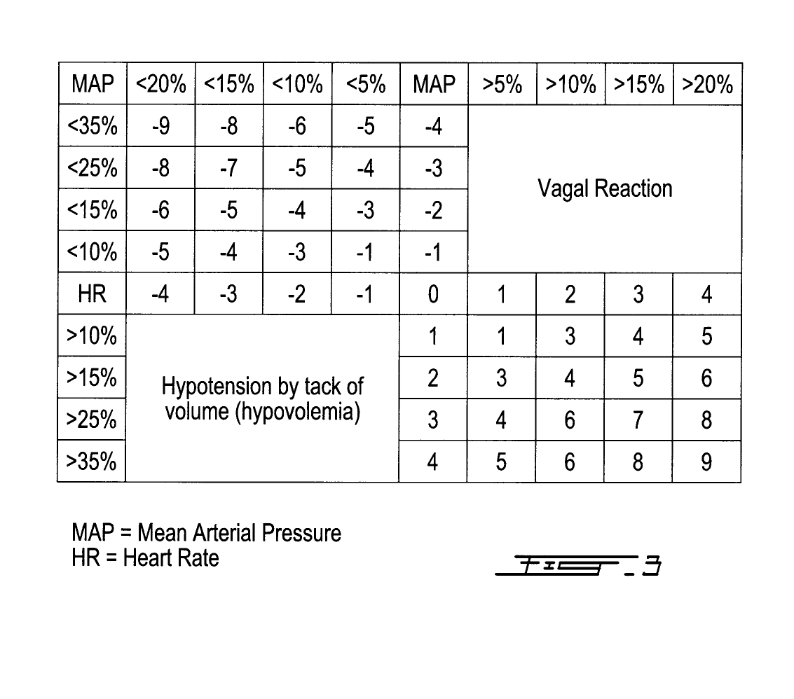

Referring now to FIG. 3 in addition to FIG. 2, the Analgoscore is obtained by comparing the offset percentage between target measured values of both MAP and HR, the target values being set by the anaesthesiologist as mentioned herein above. This method ensures that the pain level index will take into account variations between individual patients (e.g. different values of preoperative BP), as well as the various surgery-related parameters and requirements (e.g. the degree and timing of surgical stimuli). Computation of the Analgoscore involves fuzzy logic rules defined based on the anaesthesiologist's experience. In its linguistic form, fuzzy logic allows for imprecise concepts defined by a “linguistic variable” where conclusion is based on approximate information rather than precisely deducted from classical predicate logic. Using fuzzy logic, the Analgoscore is designed to range from a first level, illustratively −9, which represents excessive analgesia, to a second level, illustratively +9, which represents insufficient analgesia, in increments of 1. The control regions are defined such that −3 to +3 illustratively represents excellent pain control, -3 to +6 and +3 to +6 good pain control, and −6 to −9 and +6 to +9 insufficient pain control. The system of the present invention aims at maintaining the Analgoscore value within the excellent pain control region, i.e. between −3 and +3.

In some situations, insufficient pain control may be associated with causes other than changes in analgesia. Indeed, variations in MAP or HR can occur for reasons other than variations in the infusion level of the analgesic. For example, hypovolemia (i.e. decreased blood volume) can occur as a result of a predominant increase in HR with or without decrease in MAP. Similarly, vagal reactions (i.e. drop in blood pressure in response to emotional stimuli), which are caused by air or gas in the abdominal cavity during laparoscopic surgery (within the abdomen or pelvic cavity), are defined as a predominant decrease of HR with or without increases of MAP. In these cases, no Analgoscore is computed and the analgesic is infused at a pre-determined rate.

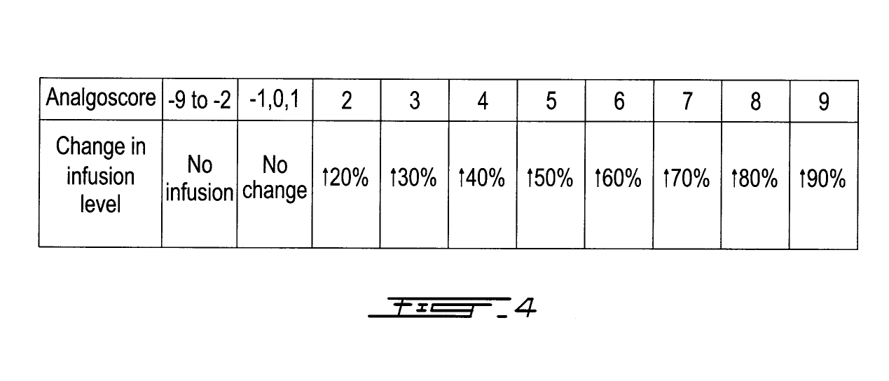

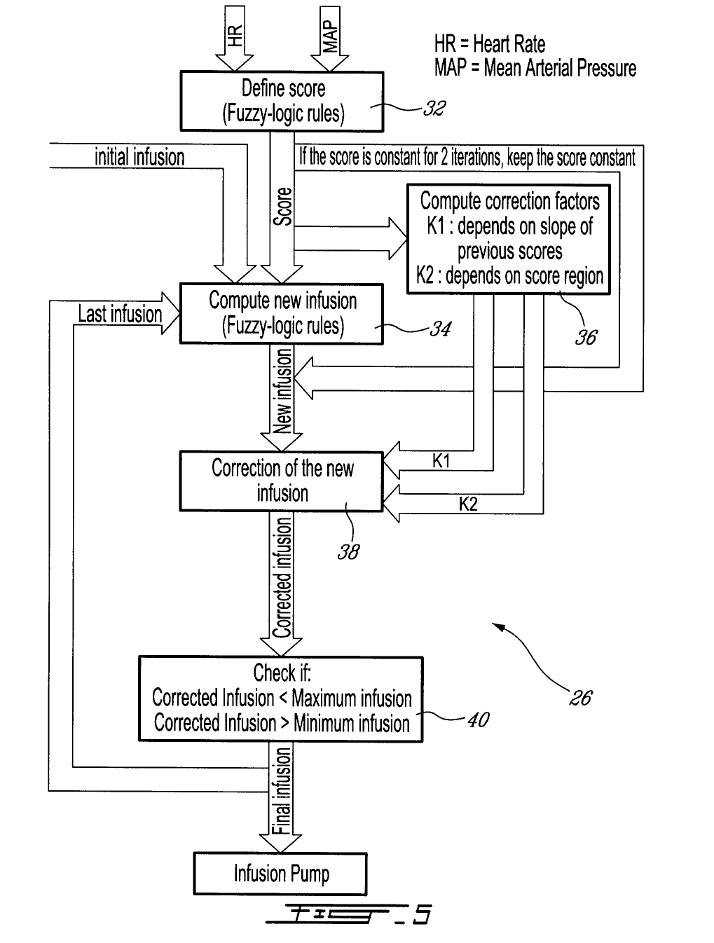

Now referring to FIG. 4 and FIG. 5 in addition to FIG. 1, once the anaesthesia control unit 26 computes the current Analgoscore from the patient's current MAP and HR (step 32), fuzzy logic rules are used at step 34 to determine the new analgesic infusion required to ease the patient's pain. Indeed, based on the current Analgoscore value, which determines whether the level of analgesia was insufficient, good or excellent, the analgesic infusion is either stopped (Analgoscore less than −2), remains the same (Analgoscore between −1 and 1) or is increased by a pre-determined percentage to reach an adequate level of analgesia. Using the control algorithm implemented by the control unit 26, if the Analgoscore remains constant for a given period of time, illustratively two consecutive minutes, the change in infusion (fuzzy-logic factor) defined in FIG. 4 is neglected, regardless of the Analgoscore value. As seen in FIG. 5, at step 36, the infusion of analgesic is illustratively further adjusted by computing two correction factors K1 and K2 in real-time, in order to take into account the evolution of the patient's state over time along with variability among patients. K1, which considers the temporal variation of the Analgoscore, is based on the average slope (“AvgSlope”) of the five previous scores. To compute K1, the slope of the scores is first computed at times t and t-2 minutes as follows:

$$Slope ( t ) = \frac{Analgoscore ( t ) - Analgoscore ( t - 2 )}{2} \qquad ( 2 )$$

The average of the previous three slopes is then computed as follows:

$$Avg Slope ( t ) = \frac{Slope ( t - 2 ) + Slope ( t - 1 ) + Slope ( t )}{3} \qquad ( 3 )$$

Computation of the average slope enables to measure the amplitude of the Analgoscore slope for the previous few minutes, illustratively the previous five minutes, as well as to minimize the effect of artefacts. A positive value of the average slope represents an augmentation of the Analgoscore and thus an augmentation of the intra-operative pain level. As a result, the infusion of analgesic will need to be increased faster. If the slope is negative, the score decreases gradually and the infusion must be reduced or even stopped completely to prevent an overdose. The value of K1 is therefore determined according to the average slope in order to specify the rate of increase or decrease of the infusion. For instance, if the score increases from −1 to 4, the infusion rate should be increased faster than if the score increases from −1 to 1. K1 is thus defined as follows:

$$K 1 = \{ \begin{matrix}2 & AvgSlope > 1 \\ 1.25 & 0.5 < AvgSlope \leq 1 \\ 1.10 & 0 < AvgSlope \leq 0.5 \\ 1 & AvgSlope = 0 \\ 0.90 & - 0.5 < AvgSlope \leq 1 \\ 0.75 & - 1 \leq AvgSlope < - 0.5 \\ - 1 & AvgSlope < - 1\end{matrix} \qquad ( 4 )$$

The second correction factor, K2, which considers the current physiological state of the patient, is based on the region within which the computed Analgoscore falls and defined as follows:

$$K 2 = \{ \begin{matrix}1.5 & 6 \leq Analgoscore < 9 \\ 1.25 & 3 \leq Analgoscore < 6 \\ 1 & 0 \leq Analgoscore < 3 \\ 0.75 & - 3 \leq Analgoscore < 0 \\ N / A & - 9 \leq Analgoscore < - 3\end{matrix} \qquad ( 5 )$$

This correction is mainly important when the slope of the Analgoscore equals zero. If the Analgoscore is between −3 and 0, the infusion rate is decreased by 25%. If the Analgoscore is between 3 and 6, the infusion rate is increased by 25% while it is increased by 50% if the Analgoscore is between 6 and 9. If the Analgoscore is between 0 and 3, K2 has no effect on the infusion.

Using the parameters described herein above, the new infusion is defined at step 38 as being the product of the previous infusion, the fuzzy-logic factor determined from FIGS. 4, K1 and K2. The control unit 26 further ensures that this corrected infusion is within an acceptable range i.e. less than a pre-determined maximal allowable infusion and greater than a pre-determined minimal infusion the anaesthesiologist wishes to maintain during surgery (step 40). If the corrected infusion is within this range, the anaesthesia control unit 26 uses it as the final infusion level and sends the information to the infusion pump 24 for administration to the patient 14. Otherwise, a new infusion will be computed starting back at step 34. The closed-loop control procedure is repeated periodically, e.g. every minute, throughout the duration of the surgery to ensure that the patient's pain level is objectively assessed and efficiently controlled.

Alternatively and as mentioned herein above, the control of the infusion pump 24 may be effected by the anaesthesiologist using his or her own judgement and experience as a tool to determine the new infusion from the Analgoscore. In this case, the present invention offers the advantage of providing an objective and quantitative measure of the state and pain level of a patient undergoing surgery.

As a result, the practitioner is able to take informed decisions based on this measure.

Referring back to FIG. 1, a mixed numerical and graphical monitoring display 30 enables integration of all three components of general anaesthesia, i.e.

hypnosis, analgesia (measured using the Analgoscore as described herein above) and neuromuscular blockade, which is representative of muscle relaxation. As mentioned herein above, neuromuscular blockade is measured using the neuromuscular function monitoring system 28, which uses a neuromuscular monitoring method such as phonomyography to record low-frequency waves generated by the spatial variations of muscles during contraction. Other methods equivalent to phonomyography, which can be used interchangeably for measuring muscle relaxation, include mechanomyography, electromyography, acceleromyography and cinemyography. As known in the art, hypnosis can be monitored through recording of auditory evoked potentials, which originate from the brain in response to an auditory stimulus, or alternatively assessed through monitoring of the BIS index. In the preferred embodiment of the present invention, data is illustratively acquired every two seconds using the BIS monitoring system 20, which continually analyses the patient's electroencephalograph (EEG) signal (measures the electrical activity of the brain) and processes it into a single number (BIS index) used to assess the patient's level of consciousness and safely predict changes in the depth of anaesthesia. The BIS index ranges from 0 to 99, with 0 being equal to EEG silence, near 100 being the expected value in a fully awake adult, and values between 40 and 60 indicating a generally accepted level for general anaesthesia.

The monitoring display 30 complements the vital signal monitoring system 18 by taking inputs from all three anaesthesia monitoring systems (i.e. the Anaesthesia control unit 26, the neuromuscular function monitoring system 28, and the BIS monitoring system 20) to present anaesthesia-related information along with important data regarding the patient's physiological state in a combination of numerical values, graphs and colours. This user-friendly integrative system reduces the anaesthesiologist's workload and eases diagnostic through better interpretation of the patient's data. It also enables effective administration of anaesthetic drugs by taking into account interactions between all three components of anaesthesia.

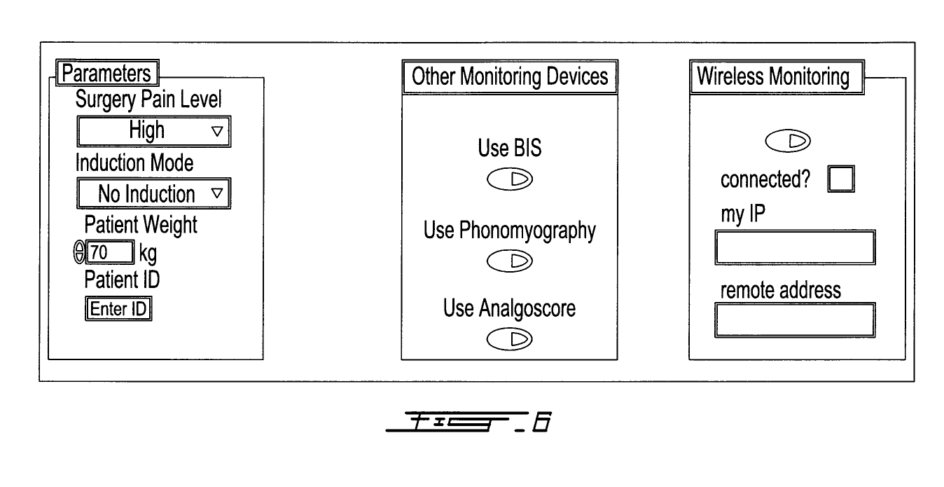

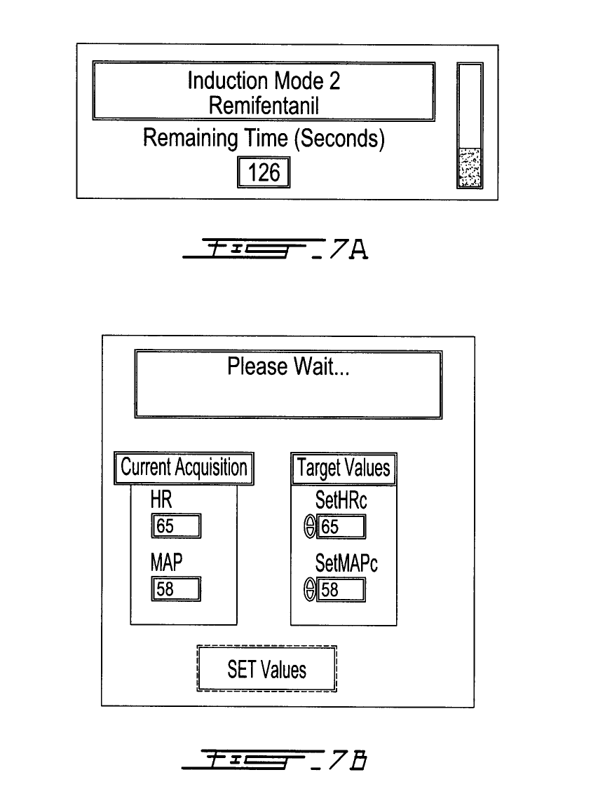

Referring now to FIG. 6, FIG. 7a and FIG. 7b, while the patient is being prepared for surgery, a setup screen or interface (see FIG. 6) is initially presented on the monitoring display 30. This setup screen enables medical staff to enter configuration parameters related to patient information such as age, weight and identification and choose the monitoring devices (e.g. Analgoscore, phonomyography, BIS, wireless monitoring) used throughout surgery for assessment of anaesthesia. Other information such as surgery pain level and anaesthesia induction mode (e.g. intravenous, inhalation) may also be entered. Once this task is completed, the monitoring display 30 illustratively displays the selected induction mode during induction as well as a progress bar and a countdown representing the time remaining until the induction is complete (see FIG. 7a). As mentioned previously herein above, target values of MAP and HR to be achieved in the patient during surgery, which are initially established by the anaesthesiologist according to the patient's health record, may subsequently be entered (FIG. 7b).

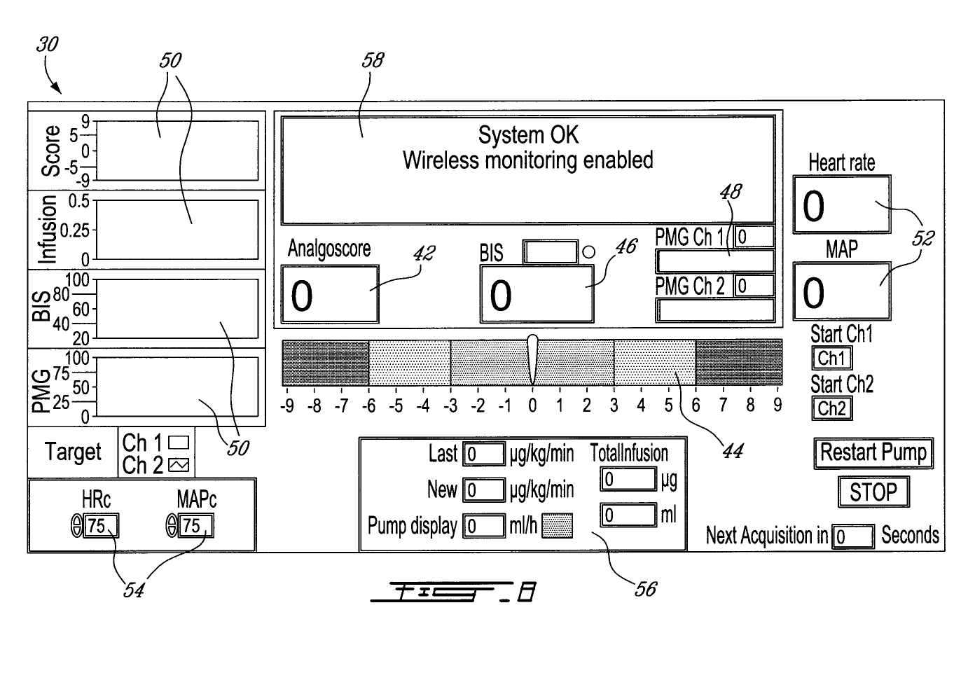

Referring now to FIG. 8, once all required data is entered, the monitoring display 30 then switches to a maintenance screen, which allows the anaesthesiologist to monitor the patient's physiological state. Illustratively, the maintenance screen is optimized to show relevant information while avoiding data overflow. It further allows for real time display as well as trend display of data for each measured physiological parameter. When data is presented in real time, for example for Analgoscore and BIS values, colour coding is used to represent the urgency of the parameters. The Analgoscore value is displayed both numerically (in field 42 of the display 30) and graphically on a horizontal bar 44 divided into green, yellow and black coloured regions, which correspond to different zones of pain control, with green indicating optimal pain control, yellow good pain control and black insufficient pain control (either too light or too profound analgesia). Similarly, the value of the BIS index (together with the signal quality) is illustratively displayed numerically (in field 46 of the display 30) using different colours depending on the urgency: yellow for a BIS index ranging from 30 to 40 and from 58 to 70, and red for a BIS index less than 30 or greater than 69. In the case of the BIS index, the red colour is reserved for situations requiring imperative attention from the anaesthesiologist. For example, red is used when values of the BIS are greater than 69, in which the anaesthesiologist must immediately adjust the level of anaesthetic agents infused since there is an imminent risk of the patient waking up. Although the green, yellow, black and red colours have been used in the preferred embodiment of the present invention, it should be understood that different colours might be used without departing from the scope of the invention.

Still referring to FIG. 8, the percentage of neuromuscular blockade is also indicated in field 48 numerically and graphically using a progress bar for up to two muscles on two separate channels (PMG Ch1 and PMG Ch2). Illustratively, the trend for each measured physiological parameter (Analgoscore, BIS index, neuromuscular blockade, infusion rate) is further displayed in fields as in 50 to allow the medical staff to follow the evolution of the surgery. The current HR and MAP are also displayed in separate fields as in 52 as well as their target values (HRc and MAPc), which appear in fields 54 and may be modified at any time during the surgery to optimally tailor the surgery to the patient. The display 30 further allows for the previous and current infusion rate (in μg/kg/min), total infusion (in both pg and ml) and pump display (in ml/h) to be represented in a separate field as in 56. The system of the present invention illustratively further provides a means (not shown) for storing the data measured during anaesthesia and displayed in the various fields of the display 30. As will be apparent to a person skilled in the art, this feature alleviates the need for manuscript notes, which are typically placed in the patient's file to assess the patient's status during surgery. Moreover, such a system further enables such data to be easily accessed and retrieved (e.g. printed) by medical staff for example when desired.

Still referring to FIG. 8 in addition to FIG. 1, an alarm system is also designed to alert the anaesthesiologist of a potential clinical or technical problem or difficulty as required for most medical monitors. Current alarm systems are often regarded as nuisance by medical staff that frequently turns them off due to a high prevalence of false alarms. In addition, some monitors allow users to customize the alarm threshold and may as a result be misleading. Indeed, when starting the device, users expect the alarms to be set at the manufacturer's default limits while they were in fact modified by a previous user. To overcome some of these and other drawbacks of traditional alarms, a descriptive message is added to the alarm sound and presented on the monitoring display 30 in a separate field 58 used for general alarm messages. If the system functions correctly and no error is detected, the descriptive message field 58 reads “System OK”. Otherwise, types of descriptive error messages include technical messages related for example to a communication error with the vital sign monitoring system 18 or physiological messages such as “Vagal reaction”, “Hypovolemia”, and “High blood pressure”. Alarm sounds that accompany alarm messages depend on the urgency of the error encountered (non-critical versus life-threatening situations) and as such, more important alarms attract the attention of the medical staff by the duration of their presence. An intermittent pattern of audible notification is used for urgent situations since it was shown to be less obstructive than a continuous sound generated once for non-critical events. For example, to alert a user of a new, non-critical alarm such as checking the BIS, a 500 Hz sound is triggered once for 100 ms. For critical errors such as a low heart rate, a 500 Hz sound is triggered every 3 s for 300 ms.

In another embodiment of the invention, remote patient monitoring is implemented, where important patient data can be transferred from the operating room to remote workstations (e.g. desktop computers or mobile computers such as tablet PCs), which are connected to the local communication network (within the hospital or clinic for example) using local network systems and protocols such as the Transmission Control Protocol/Internet Protocol (TCP/IP). Since the anaesthesiologist is still responsible of indirect patient care and monitoring outside the operating room and needs a complete description of the anaesthesia currently in progress, patient data can also be transferred to a mobile communication module (e.g. a Personal Digital Assistant (PDA)) carried by the anaesthesiologist. In this case, a communication system is illustratively implemented between the mobile communication module and the operating room. To acquire data, the user only needs to setup a wireless communication between the operating room computer and the mobile device without the need for any further assistance. Such a mobile solution therefore fits into the anaesthesiologist's workflow while offering the advantages of real time access to data for the patient currently undergoing surgery and better communication with the operating room, using text messaging for example. Any wireless communication protocol can be used to implement communications with the mobile device, including custom designed protocols or standards such as Bluetooth and Wireless Fidelity (Wi-Fi). However, Wi-Fi technology is preferably chosen since its communication range can be widened according to the needs of the application, unlike Bluetooth whose maximum range is about 10 m. In addition, to prevent patient data transmitted wirelessly to the mobile device from being hacked, security measures such as encryption and firewalls are implemented. Since an exact duplication of the monitoring display interface used in the operating room onto a mobile communication device interface is not typically possible, the mobile device interface typically relies more on numeric data than on graphical displays. Still, the alarm sound generated in case of emergency on the mobile device will have the same frequency and duration as the one used in the main monitoring display interface but with higher amplitude to cover ambient noise, which is higher outside of the operating room.

Although the present invention has been described hereinabove by way of specific embodiments thereof, it can be modified, without departing from the spirit and nature of the subject invention as defined in the appended claims.

1. A method for displaying an indicator of a current pain level of a patient being administered an analgesic, the method comprising:
providing a display device;
measuring a current mean arterial pressure and heart rate of the patient;
deriving the indicator from said measured current mean arterial pressure and heart rate; and
displaying said derived indicator on said display device.

2. The method of claim 1, wherein said derived indicator is displayed on said display device as numerical data, graphical data, colour coding and combinations thereof.

3. The method of claim 1, wherein desired target values of said mean arterial pressure and said heart rate are determined prior to administering said analgesic and said deriving an indicator comprises comparing said current mean arterial pressure and heart rate to said target values using fuzzy logic rules.

4. The method of claim 1, wherein said derived indicator is defined in a range from a first level to a second level, said first level representing an excessive analgesia level and said second level representing an insufficient analgesia level.

5. The method of claim 4, wherein said range comprises a plurality of predetermined regions, at least a first one of said predetermined regions representing inadequate pain control, at least a second one of said predetermined regions representing good pain control, and at least a third one of said predetermined regions representing excellent pain control.

6. The method of claim 5, further comprising calculating a change in rate of infusion based on said derived indicator, recalculating said change in rate of infusion based on an average change in said derived indicator over time and a current value of said derived indicator, and adjusting a rate of infusion of the analgesic according to said recalculated change in rate of infusion.

7. The method of claim 6, wherein said calculating a change in rate of infusion comprises maintaining said infusion, stopping said infusion, or increasing said infusion according to said predetermined region said derived indicator lies in.

8. The method of claim 6, wherein said recalculating said change in rate of infusion comprises computing a first correction factor representative of a temporal variation of said derived indicator and a second correction factor representative of a current physiological state of the patient and applying said first and second correction factors to said calculated change in rate of infusion.

9. A system for displaying an indicator representative of a current pain level of a patient being administered an analgesic, the system comprising:
a monitoring subsystem for measuring a current mean arterial pressure and heart rate of the patient and deriving the indicator from said measured mean arterial pressure and heart rate; and
a display device coupled to said monitoring subsystem for displaying said derived indicator.

10. The method of claim 9, wherein said display device displays said derived indicator using numerical data, graphical data, colour coding, and combinations thereof.

11. The system of claim 9, wherein said monitoring subsystem comprises a vital sign monitoring system for measuring said current mean arterial pressure and heart rate of the patient.

12. The system of claim 9, further comprising a delivery subsystem coupled to said monitoring subsystem for administering the analgesic to the patient, wherein said monitoring subsystem adjusts a rate of infusion of the analgesic according to said derived indicator and outputs said adjusted rate of infusion to said delivery subsystem.

13. The system of claim 12, wherein said delivery subsystem is an infusion pump.

14. The system of claim 12, wherein said monitoring subsystem adjusts said rate of infusion by calculating a change in rate of infusion based on said derived indicator, recalculating said change in rate of infusion based on an average change in said derived indicator over time and a current value of said derived indicator, and adjusting said rate of infusion according to said recalculated change in rate of infusion.

15. The system of claim 14, wherein desired target values of said mean arterial pressure and said heart rate are determined prior to administering the analgesic and said monitoring subsystem derives the indicator by comparing said current mean arterial pressure and heart rate to said target values using fuzzy logic rules.

16. The system of claim 15, wherein said derived indicator is defined in a range from a first level to a second level, said first level representing an excessive analgesia level and said second level representing an insufficient analgesia level.

17. The system of claim 16, wherein said range comprises a plurality of predetermined regions, at least a first one of said predetermined regions representing inadequate pain control, at least a second one of said predetermined regions representing good pain control, and at least a third one of said predetermined regions representing excellent pain control.

18. The system of claim 17, wherein said monitoring subsystem calculates said change in rate of infusion by maintaining the infusion, stopping the infusion, or increasing the infusion according to said predetermined region said derived indicator lies in.

19. The system of claim 14, wherein said monitoring subsystem recalculates said change in rate of infusion by computing a first correction factor representative of a temporal variation of said derived indicator and a second correction factor representative of a current physiological state of the patient and applying said first and second correction factors to said calculated change in rate of infusion.

20. The system of claim 12, wherein a minimum and a maximum rate of infusion defining a desired range of infusion are determined prior to administering the analgesic and further wherein said monitoring subsystem compares said adjusted rate to said minimum and said maximum rate of infusion and outputs said adjusted rate to said delivery subsystem if said adjusted rate lies within said desired range.

21. A system for displaying a current state of anaesthesia of a patient undergoing surgery, the system comprising:
a first subsystem for measuring a current anaesthetic depth in the patient;
a second subsystem for monitoring a current level of muscular relaxation in the patient;
a third subsystem for deriving an indicator representative of a current pain level of the patient; and
a single display device coupled to said first, second, and third subsystems for simultaneously displaying said current anaesthetic depth, said current level of muscular relaxation and said derived indicator.

22. The system of claim 21, wherein said first subsystem measures said current anaesthetic depth using a method selected from the group consisting of monitoring auditory evoked potentials produced by the patient in response to repetitive audio stimulus, monitoring the bispectral index of the patient, spectral entropy, and combinations thereof.

23. The system of claim 21, wherein said second subsystem monitors said current level of muscular relaxation using a method selected from the group consisting of phonomyography, mechanomyography, electromyography, acceleromyography, cinemyography, and corn binations thereof.

24. The system of claim 21, wherein said third subsystem comprises a vital sign monitoring system for measuring a current mean arterial pressure and heart rate of the patient, further wherein said third subsystem derives said indicator from said measured current mean arterial pressure and heart rate.

25. The system of claim 21, wherein said third subsystem adjusts according to said derived indicator a rate of infusion of an analgesic being administered to the patient to achieve general anaesthesia in the patient.

26. The system of claim 24, wherein said display device is further coupled to said vital sign monitoring system to display said current mean arterial pressure and heart rate.

27. The system of claim 21, wherein said display device displays a first interface for entering configuration parameters comprising of identification information of he patient, a weight of the patient, an age of the patient, an induction mode of said analgesic, a pain level of the surgery, and combinations thereof.

28. The system of claim 27, wherein subsequently to displaying said first interface, said display device displays a second interface representing said current anaesthetic depth, said current level of muscular relaxation and said derived indicator.

29. The system of claim 28, wherein said current anaesthetic depth, said current level of muscular relaxation and said derived indicator are represented on said second interface using numerical data, graphical data, colour coding, and combinations thereof.

30. The system of claim 21 further comprising an alarm subsystem for alerting of at least one difficulty related to the surgery, said alarm subsystem emitting at least one of a plurality of alarm sounds and displaying at least one of a plurality of descriptive messages according to said at least one difficulty.

31. The system of claim 30, wherein according to the urgency of said difficulty said at least one of a plurality of alarm sounds varies in intensity, duration, pattern, and combinations thereof.

32. The system of claim 21, wherein said display device is at least one of a plurality of remote workstations and is coupled to said first, second, and third subsystems via a local communications network.

33. The system of claim 32, wherein said plurality of remote workstations comprises of desktop computers, mobile computers, and mobile communication modules.

34. The system of claim 32, wherein data related to said current anaesthetic depth, said current level of muscular relaxation and said derived indicator is transmitted wirelessly to said at least one of said plurality of remote workstations.
